(19) United States
(12) Patent Application Publication
(10) Pub. No.: US 2022/0296873 A1
(43) Pub. Date: Sep. 22, 2022

(54) CONNECTOR ASSEMBLY FOR USE IN MEDICAL FLUID LINES

(71) Applicant: David Liu, Mequon, WI (US)

(72) Inventor: David Liu, Mequon, WI (US)

(21) Appl. No.: 17/701,376

(22) Filed: Mar. 22, 2022

Related U.S. Application Data
(60) Provisional application No. 63/207,778, filed on Mar. 22, 2021.

(51) Int. Cl. A61M 39/18; F16L 37/46; F16L 37/12; A61M 39/16

(52) U.S. Cl. CPC A61M 39/18; F16L 37/46; F16L 37/12; A61M 39/165; F16L 2201/44

(57) ABSTRACT

A connector assembly for connection and disconnection between ports of fluid lines for administering fluids into a human body from a route other than the oral route. The connector assembly includes a catheter dock, a cap removal dock, a fluid line dock, and a new cap dock. The catheter dock can receive an inlet port of a catheter secured by a catheter cap. The cap removal dock can aseptically remove the catheter cap from the inlet port. The catheter dock can be switched consecutively between the cap removal dock, the fluid line dock, and the new cap dock. Upon removal of the catheter cap, the catheter dock can be switched to the fluid line dock, and the fluid can flow from the fluid line to the catheter. Upon administering the liquid, the catheter dock can be switched from the fluid line dock to the new cap dock, wherein a new catheter cap is secured into the inlet port under aseptic conditions.

### CROSS-REFERENCE TO RELATED APPLICATIONS

This application claims priority from the U.S. provisional patent application Ser. No. 63/207,778, filed on Mar. 22, 2021, which is incorporated herein by reference in its entirety.

### FIELD OF THE INVENTION

The present invention relates to a connector assembly for medical fluid lines, and more particularly, the present invention relates to a connector assembly to connect and disconnect medical fluid lines in a sterilized manner.

### BACKGROUND

Administering fluid into the body of a human or animal, from a route other than the oral route, is common in a variety of treatments and therapies, such as fluid replacement therapy, fluid irrigation, and the like. Typically, to administer the fluid, a cannula or catheter is inserted into an organ or a cavity of the body. The fluid is contained in a reservoir, and a fluid line connects the reservoir and the cannula or the catheter. The cannula, catheter, or any fluid line that is used to administer the fluid into the body of a human or animal are referred to herein as a catheter. Some examples of therapies that fit this description include chemotherapy infusions for cancer; hemodialysis and peritoneal dialysis for end-stage-renal-disease; total parenteral nutrition for ulcerative colitis and Crohn's disease; intravenous infusions for various diseases and conditions.

In most of the treatments, the catheter is inserted into the bloodstream or another vulnerable area of the human body. For example, central venous catheters (CVCs) used in chemotherapy and emergency hemodialysis are surgically implanted into a patient's internal jugular vein which leads directly to the heart. Catheters for peritoneal dialysis are implanted into the patient's peritoneal cavity, an area of the human body highly susceptible to infections. Subsequently, penetration of microbes and other contaminants into the catheter tubing may result in a serious or deadly infection episode. Thus, the healthcare professionals (and sometimes patients for home-therapies) must take extraordinary precautions and comply with aseptic protocols when connecting and disconnecting fluid lines.

Catheters, fluid lines, and reservoirs containing the fluids are manufactured, packed, and sold separately. The catheters and the fluid lines have ports for fluidly connecting the catheters to the fluid line and the fluid line to the reservoir. The ports are generally covered by caps to maintain sterility, wherein the caps can be removed for connecting the catheter to the fluid line, and after use, the caps can be replaced to prevent exposure to contaminants.

However, the present apparatuses and methods for connecting and disconnection of the caps suffer from many drawbacks that increase the exposure to germs and contaminants and the risk for an infection episode. For the time, the ports are in open state, the fluid pathway becomes vulnerable to microbes and contaminants.

It has been proposed to disinfect the ports, for example, U.S. patent Ser. No. 10/166,085 B2 granted to Hub Hygiene and U.S. patent Ser. No. 10/279,058 B2 granted to Ultraviolet Interventions, Inc. teaches methods and apparatuses for disinfecting fluid line ports and catheter ports. However, the known apparatuses and methods for disinfecting the ports utilize an external device. Such methods and apparatuses do not properly address the element of human error, which is the principal driver of contamination events. Human error occurs in the form of negligence in failing to follow aseptic technique or disinfectant protocols, or accidental contact between a sterile portion of the port and a non-sterile surface. The known system and method for maintaining sterility of ports, and thus the fluid lines and catheters do not provide a complete solution, and a risk of some source of contamination exists.

Thus, a need is appreciated for an assembly and method that allow connecting and disconnecting ports of the catheter and the fluid line in a sterile manner and maintains sterility during use and after use.

### SUMMARY

The principal object of the present invention is therefore directed to a connector assembly and method for connecting and disconnecting ports of a catheter and a fluid line in a sterile manner.

It is another object of the present invention that the connector assembly is easy to use.

It is still another object of the present invention that the connector assembly can be single-use and disposable.

It is yet another object of the present invention that the connector assembly can remain sterile between the connection and disconnection of the catheter and the fluid line.

In one aspect, disclosed is a connector assembly that can be designed and used for connecting fluid lines to multi-use connector ports. The disclosed connector assembly can be used for catheters and fluid lines that require the placement of a new protective cap after each use. The disclosed assembly can permit a user to perform the process of port connection and disconnection within a sterilized enclosed environment. Moreover, the disclosed assembly and method may eliminate human error as a risk factor for contamination. The disclosed connector assembly can assist a user in performing the entire connection and disconnection process i.e., removal of safety cap, connecting and disconnecting fluid channels, and placement of a new cap without error each time. The inlet port of the catheter, the caps, and the port of the fluid lines are always enclosed within a sterilized body of the disclosed connector assembly, mitigating the risk of room air or surface contaminants contacting and breaching the fluid pathway.

In one aspect, disclosed is connector assembly for aseptically connecting and disconnecting a fluid line to a catheter, the connector assembly comprises a catheter dock, the catheter dock comprising a first housing configured to receive an inlet port of a catheter, the inlet port of the catheter has an opening, the opening secured by a catheter cap; and a locking member configured to secure the inlet port within the catheter dock; a cap removal dock comprising a second housing; a first rod rotatably and slidably received into the second housing, the first rod has a mouth, the mouth of the first rod configured to releasably couple to the catheter cap while the catheter cap is fastened to the inlet port of the catheter; and a fluid line dock comprising a third housing; a valve unit encased within the third housing, the valve unit is in fluid communication with a fluid line, wherein the valve unit is configured to switch between an open state and a closed state, fluid from the fluid line flows freely from the valve unit in the open state, the fluid does not flow from the valve unit in the closed state, wherein catheter dock is aligned with the cap removal dock such that the catheter cap can be removed by the first rod, wherein the catheter dock is configured to aseptically switch from the cap removal dock to the fluid line dock, wherein the fluid line dock is configured to be actuated to fluidly couple the valve unit and the inlet port of the catheter, while switching the valve unit from the closed state to the open state, resulting in a fluid communication between the fluid line and the catheter, wherein the fluid line dock is configured to be separated from the inlet port of the catheter resulting in switching of the value unit from the open state to the closed state.

In one implementation, the connector assembly further comprises a new cap dock, the new cap dock comprises a fourth housing; a second rod slidably and rotatably received within the fourth housing, the second rod has a mouth; and a new catheter cap releasably coupled to the mouth of the second rod, wherein the catheter dock is configured to aseptically switch from the fluid line dock to the new cap dock such that the new cap dock and the catheter dock are aligned with each other, wherein the new cap dock is configured to be actuated for fastening the new catheter cap to the inlet port of the catheter.

In one implementation, the connector assembly further comprises a front planar member, the front planar member has a front face and a rear face; a rear planar member, the rear planar member has a front face and a rear face, the front planar member is rotatably mounted to the rear planar member, wherein the rear face of the front planar member faces the front face of the rear planar member, wherein the first housing extends perpendicular from the front face of the front planar member, wherein the second housing extends perpendicular from the rear face of the rear planar member, wherein the third housing extends perpendicular from the rear face of the rear planar member, the second housing and the third housing are spaced apart from each other, wherein the front planar member is configured to be rotated relative to the rear planar member for aseptically switching the catheter dock between the cap removal dock and the fluid line dock.

In one implementation, the connector assembly further comprises a front planar member, the front planar member has a front face and a rear face; a rear planar member, the rear planar member has a front face and a rear face, the front planar member is rotatably mounted to the rear planar member, wherein the rear face of the front planar member faces the front face of the rear planar member, wherein the first housing extends perpendicular from the front face of the front planar member, wherein the second housing extends perpendicular from the rear face of the rear planar member, wherein the third housing extends perpendicular from the rear face of the rear planar member, wherein the fourth housing extends perpendicular from the rear face of the rear planar member, the second housing, the third housing, and the fourth housing are spaced apart from each other, wherein the front planar member is configured to be rotated relative to the rear planar member for aseptically switching the catheter dock from the cap removal dock to the fluid line dock, and from the fluid line dock to the new cap dock.

In one implementation, the connector assembly further comprises a male fastening member and a female fastening member, wherein the male fastening member and the female fastening member are at the centers of the front planar member and the rear planar member for rotatably coupling the front planar member to the rear planar member.

In one implementation, the male fastening member and the female fastening member are configured to halt rotation of the front planar member relative to the rear planar member at predefined intervals.

In one implementation the male fastening member and the female fastening member are configured to limit the rotation of the front planar member relative to the rear planar member in one direction.

In one implementation the predefined intervals are at 120 degrees.

In one implementation the front planar member is sealably coupled to the rear planar member.

In one implementation the first housing is integral with the front planar member, wherein the second housing, the third housing, and the fourth housing are integral with the rear planar member.

In one implementation the connector assembly further comprises a pin lock comprising one or more pins, wherein the first housing has one or more apertures for the one or more pins to pass through into one or more apertures in the inlet port of the catheter.

In one implementation, the valve unit comprises a valve tip; and a stem tip, the stem tip is in fluid communication with the fluid line, wherein the valve tip and the step-tip form a spool valve.

In one aspect, disclosed is a method for aseptically connecting and disconnecting a fluid line to a catheter, the method comprising the steps of providing a connector assembly, inserting an inlet port of the catheter into the catheter dock; upon inserting, securing the inlet port within the catheter dock by the locking member; removing the catheter cap from the inlet port by turning the first rod to unfasten the catheter cap and upon unfastening the catheter cap, pulling the first rod to draw the catheter cap into the second housing; upon removal of the catheter cap, switching the catheter dock to the fluid line dock; and upon switching, when the catheter dock is aligned with the fluid line dock, actuating the fluid line dock to fluidly couple the valve unit and the inlet port causing the switching of the valve unit from the closed state to the open state and flow of the fluid from the fluid line to the catheter.

In one implementation, the method further comprises the steps of upon completion of administration of the fluid, separating the valve unit from the inlet port; and upon separating, switching the catheter dock to the new cap dock; and fastening, the new catheter cap to the inlet port.

### DRAWINGS

Some embodiments of the presented invention are illustrated as an example and are not limited by the figures of the accompanying drawings, in which like references may indicate similar elements and in which:

FIG. 1 is a perspective view of the connector assembly with a catheter, according to an exemplary embodiment of the present.

FIG. 2 is a perspective view of the connector assembly without the catheter, according to an exemplary embodiment of the present.

FIG. 3 is another perspective view of the connector assembly without the catheter, according to an exemplary embodiment of the present.

FIG. 4 is another perspective view of the connector assembly without the catheter, according to an exemplary embodiment of the present.

FIG. 5 is a perspective view of the connector assembly with the catheter showing the attachment of the inlet port of the catheter into a catheter dock of the connector assembly, according to an exemplary embodiment of the present.

FIG. 6 is an exploded view of the connector assembly of FIG. 1, according to an exemplary embodiment of the present.

FIG. 7 is an exploded view of the connector assembly of FIG. 2, according to an exemplary embodiment of the present.

FIG. 8 shows an inlet port of the catheter and a cap of the catheter, according to an exemplary embodiment of the present invention.

FIG. 9 is a rear view of the front member, showing a disc having an aperture that is positioned off-center and a mating member at the center of the disc, according to an exemplary embodiment of the present invention.

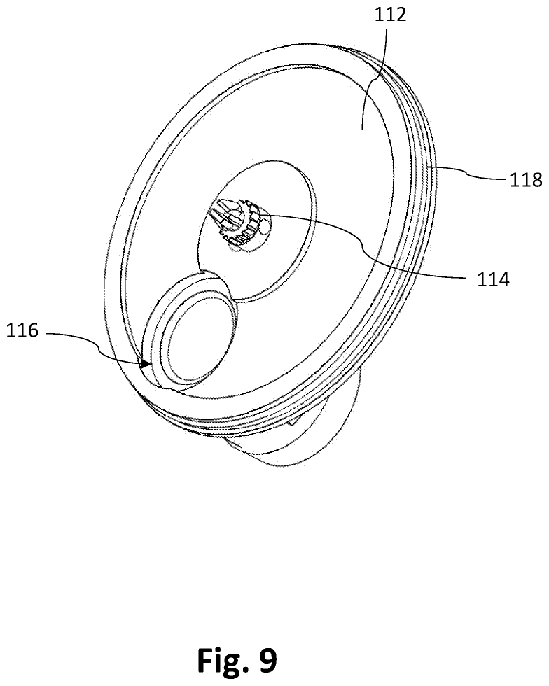

FIG. 10 is an exploded view of the front member of the connector assembly showing the disc, the catheter dock, a lock, and locking pins set, according to an exemplary embodiment of the present invention.

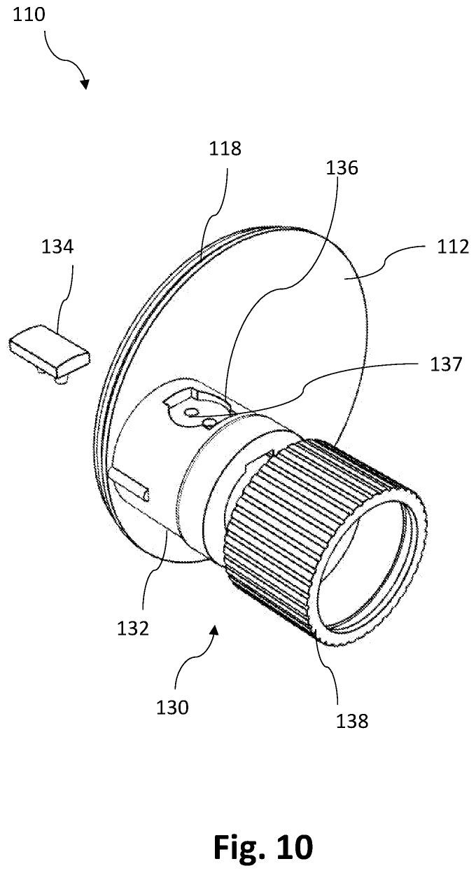

FIG. 11 is a side view of the front member, according to an exemplary embodiment of the present invention.

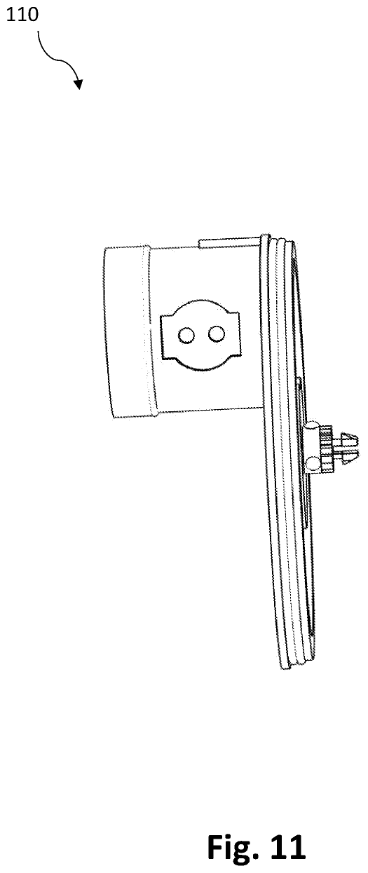

FIG. 12 is a front and side perspective view of the connector assembly having the disc and a housing of the catheter dock both hidden, according to an exemplary embodiment of the present invention.

FIG. 13 shows the base of the rear member having the three housing/chambers of the cap removal dock, fluid line dock, and new cap dock, according to an exemplary embodiment of the present invention.

FIG. 14 is another view of the base, according to an exemplary embodiment of the present invention.

FIG. 15 is a rear and side perspective view of the connector assembly having the base and three housings of the cap removal dock, fluid line dock, and new cap dock hidden, according to an exemplary embodiment of the present invention.

FIG. 16 shows the cap removal dock, fluid line dock, and the new cap dock without the housings and the base of the rear member, according to an exemplary embodiment of the present invention.

FIG. 17 is a front and side perspective view of the rear member of the connector assembly, according to an exemplary embodiment of the present invention.

FIG. 18 is a perspective view of a cap removal dock of the rear member, according to an exemplary embodiment of the present invention.

FIG. 19 is a perspective view of a fluid line dock of the rear member, according to an exemplary embodiment of the present invention.

FIG. 20 is an exploded view of the fluid line dock of FIG. 19, according to an exemplary embodiment of the present invention

FIG. 21 is a perspective view of the new cap dock, according to an exemplary embodiment of the present invention.

FIG. 22 is an exploded view of the new cap dock of FIG. 21, according to an exemplary embodiment of the present invention.

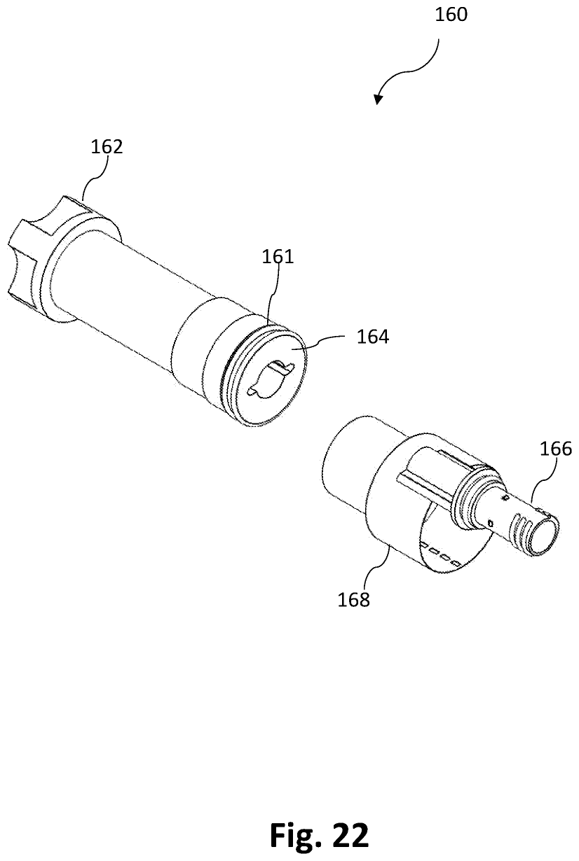

### DETAILED DESCRIPTION

The terminology used herein is to describe particular embodiments only and is not intended to be limiting of the invention. As used herein, the term “and/or” includes any and all combinations of one or more of the associated listed items. As used herein, the singular forms “a,” “and,” and “the” are intended to include the plural forms as well as the singular forms, unless the context clearly indicates otherwise. It will be further understood that the terms “comprises” and/or “comprising,” when used in this specification, specify the presence of stated features, steps, operations, and/or components, but do not preclude the presence or addition of one or more features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the presented disclosure and will not be interpreted in an idealized or overly formal sense unless expressly so defined herein.

In describing the invention, it will be understood that a number of techniques and steps are disclosed. Each of these has individual benefits and each can also be used in conjunction with one or more, or in some cases all, of the other disclosed techniques. Accordingly, this description refrains from repeating every possible combination of the individual steps in an unnecessary fashion. Nevertheless, the specification and claims should be read with the understanding that such combinations are entirely within the scope of the invention and the claims.

The present disclosure is to be considered as an exemplification of the invention and is not intended to limit the invention to the specific embodiments illustrated by the figures or description below. While preferred materials for elements have been described, the device is not limited by these materials. Wood, plastic, rubber, foam, metal alloys, aluminum, and other materials may comprise some or all of the elements of the device.

The present invention will now be described by referencing the appended figures representing preferred embodiments of the device.

Disclosed is a connector assembly for use in the medical field for connecting medical fluid lines used for administering fluids into a body of a human or animal by a route other than an oral route. Disclosed is a disposable connector assembly that allows fluidly connecting two fluid lines in a sterilized manner. For example, a fluid line extending from a fluid reservoir can be connected and disconnected from a catheter in a sterilized manner through the disclosed connector assembly. It is however understood that certain embodiments have been described using a catheter as the fluid line, however, any other fluid line known to a skilled person for administering fluids directly into a body of a human or an animal is within the scope of the present invention.

In certain embodiments, the disclosed connector assembly can be a single-use disposable assembly. The connector assembly can be manufactured from medical-grade plastic materials, such as but not limited to polyethylene. The connector assembly can be sterilized and packaged in suitable packaging that can maintain the sterility of the disclosed connector assembly till use. Sterilization of plastic medical devices such as the infusion sets is known, and any such sterilization method can be used for sterilization of the disclosed connector assembly without departing from the scope of the present invention.

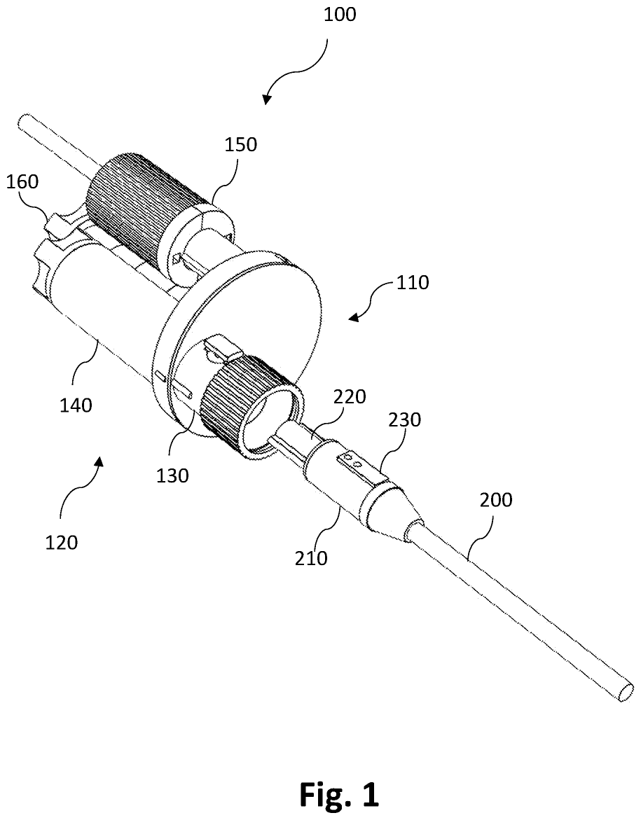

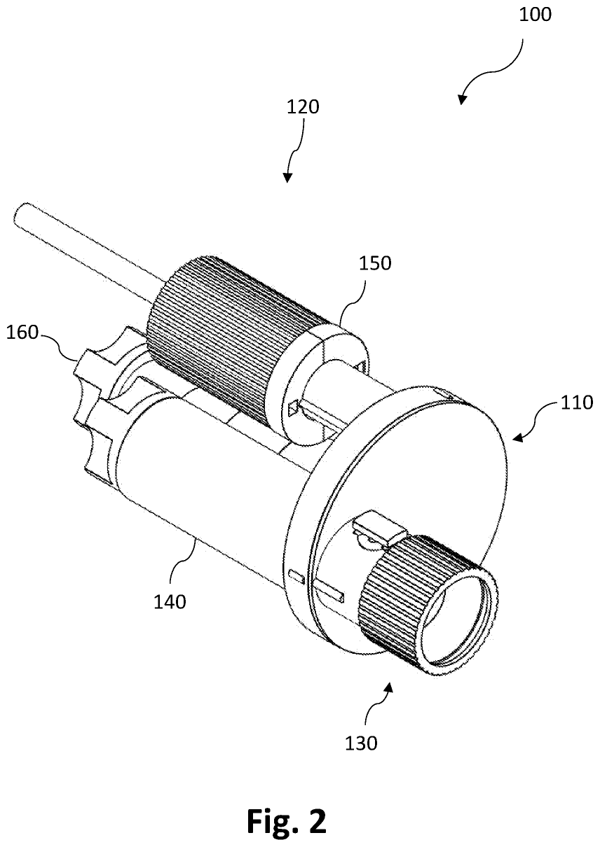

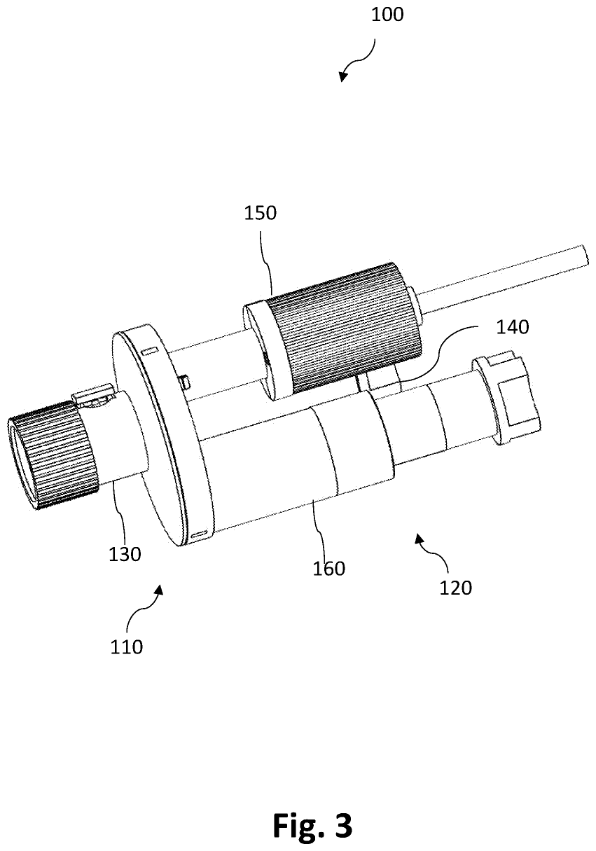

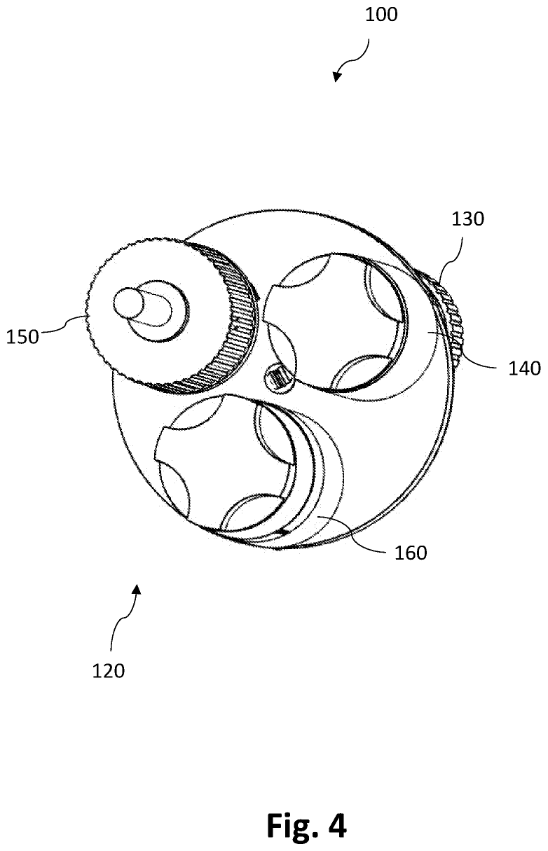

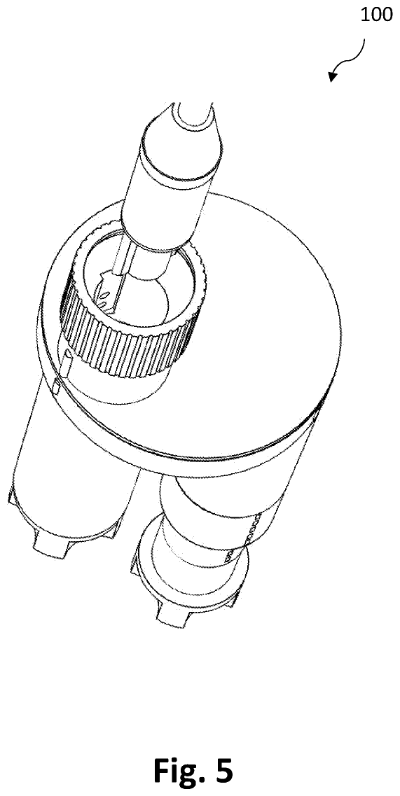

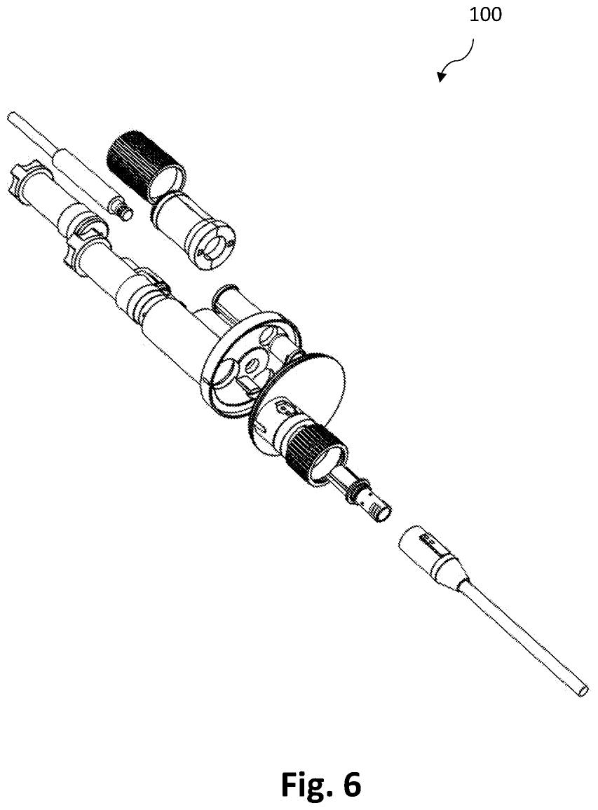

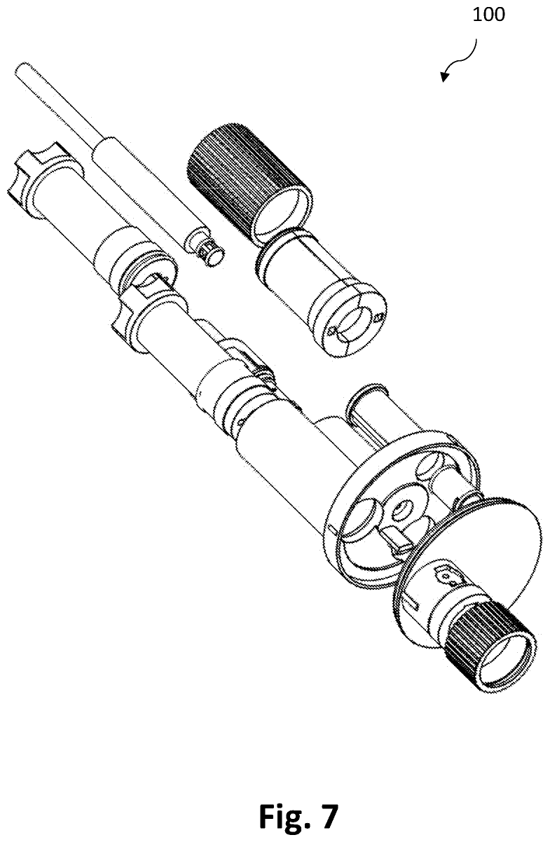

Referring to the drawings, FIG. 1 shows the disclosed connector assembly 100 and a catheter 200, the catheter has an inlet port, the opening of which is secured by a cap 220. FIGS. 2-4 show different views of the connector assembly 100 without the catheter. FIG. 5 illustrates the attachment of the catheter into a catheter dock of the connector assembly 100. FIG. 6 shows an exploded view of the connector assembly of FIG. 1. FIG. 7 is an exploded view of the connector assembly of FIG. 2.

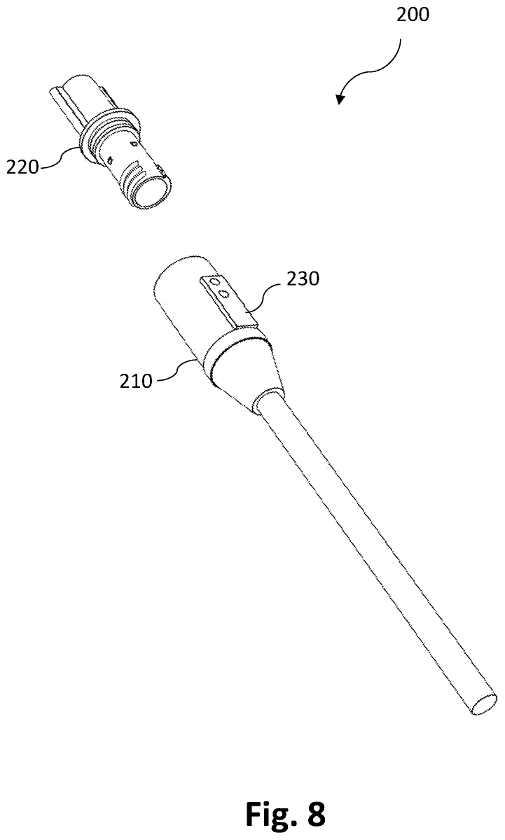

The catheter 200 is more clearly shown in FIG. 8 can include an inlet port 210 for connecting to a fluid line. The opening of the inlet port 210 can be protected by a cap 220. The structure and shape of the cap are shown in FIG. 8. A pair of apertures 230 can be used to lock the catheter in place using the locking pins/pin lock set of the disclosed connector assembly 100.

Again, referring to FIG. 2, the connector assembly 100 can include a front member 110 and a rear member 120. The front member 110 can be rotatably coupled to the rear member 120. The front member 110 can include a catheter dock 130 on a front side of a disc 112. The disc can be planar as shown in the drawings with a mating member 114 on the rear side of the disc 112. The mating member 114 can rotatably fasten the disc 112 to the rear member 120. The front member can rotate relative to the rear member at fixed intervals, such as 120 degrees or 180 degrees. The mating member can have a shaft that fits into the female mating member in the housing of the rear member. The mating member may permit the rotation of the front member relative to the rear member only in a single direction, such as clockwise. Suitable stops can be provided, such as at 120 degrees or 180 degrees i.e., when the catheter docks consecutively align with the cap removal dock, fluid line dock, and the new cap dock. In case, there are only the cap removal dock and the fluid line dock, the rotation can be stopped at two intervals, such as at 180 degrees.

The disc 112 can have an aperture 116 that can be positioned off-center. The size of the aperture can be at least more than the diameter of the cap of the catheter. Over the aperture on the front side of the disc can be coupled a catheter dock 130.

The catheter dock 130 can receive the inlet port 210 of the catheter 200. At the aperture, can be provided an O-ring 118 or similar sealing member that can sealable secure the inlet port of the catheter. The 0 ring can also prevent unnecessary movement of the inlet port of the catheter. The catheter dock has a cylindrical housing 132 which receives and encases the inlet port of the catheter. After inserting the catheter into the catheter dock, the user can push the locking pins until a click sound can be heard, the cylindrical housing can include a recess 136 in a wall of the cylindrical housing. A pair of apertures 137 in the wall and within the recess can also be seen. An outer surface of the cylindrical housing can be provided with mating threads.

The catheter dock can further include a pin lock 134. The head of the pin lock 134 can fit into the recess 136 and the pins can insert into the apertures 137. It is understood, however, that number of apertures and the pins can be varied. The locking pins can insert into the apertures 230 in the inlet port of the catheter, for securing the catheter within the catheter dock. The catheter dock can further include a twist lock 138 that can have mating threads, wherein the twist-lock can fasten over the cylindrical housing 132. The twist-lock can secure the locking pins in place, and prevent any accidental pull out of the pins. It is to be noted that the catheter can be secured into the catheter dock by any other fastening means and any such other fastening means is within the scope of the present invention. O-rings can also be provided between the cylindrical housing and the twist-lock to further secure the twist lock. When not in use, the opening of the catheter dock can be covered by a cap, the cap can be removed for docking the catheter and can be replaced after the catheter is removed. Alternatively, a breakable seal can secure the opening of the catheter dock.

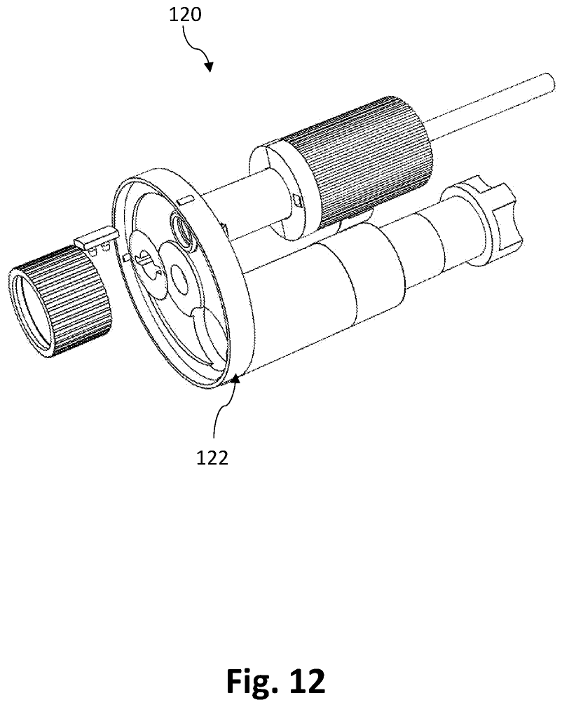

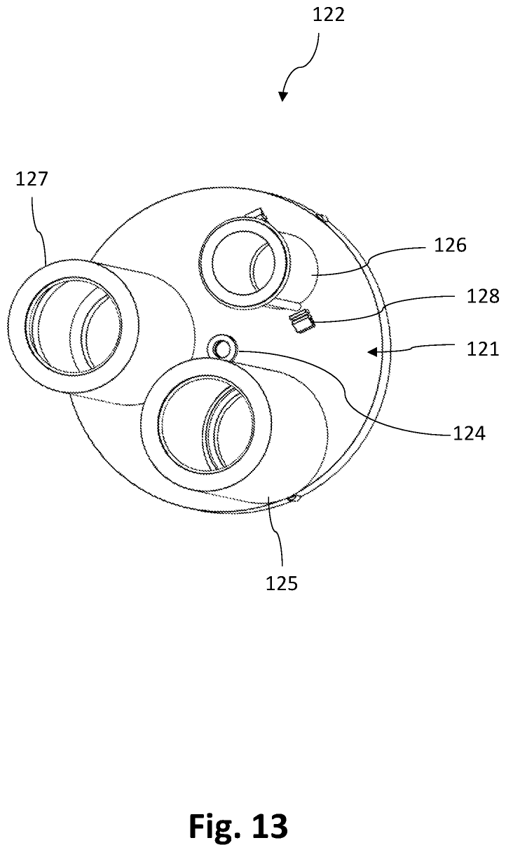

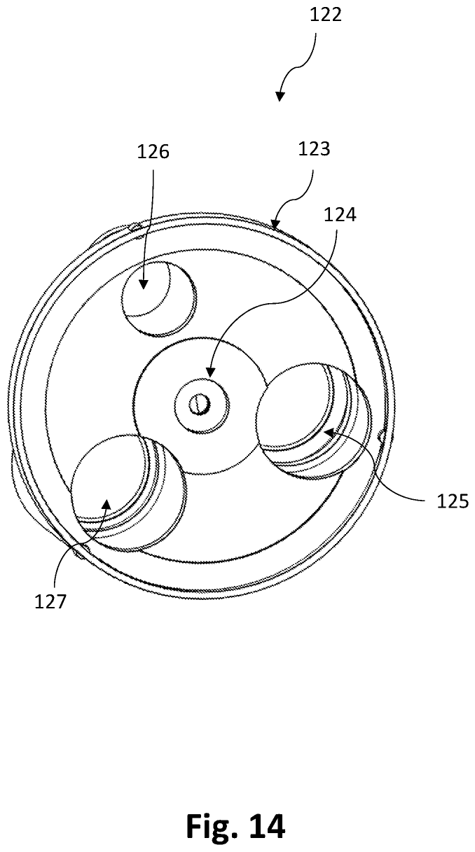

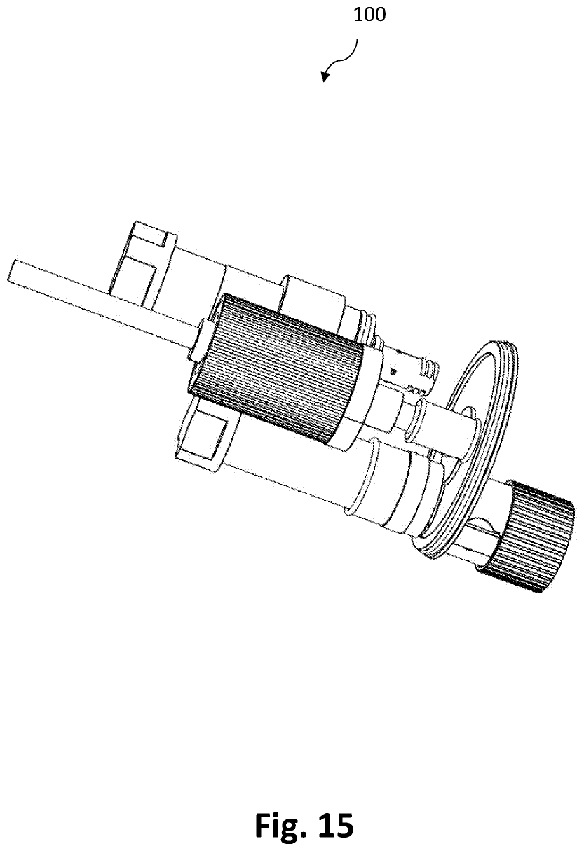

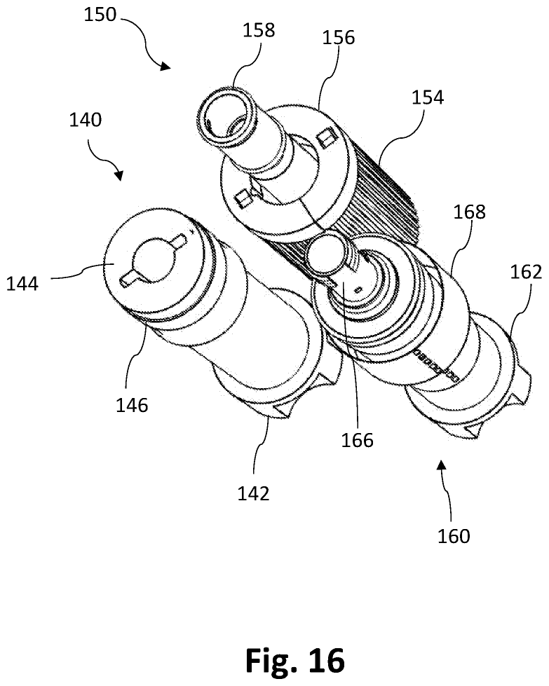

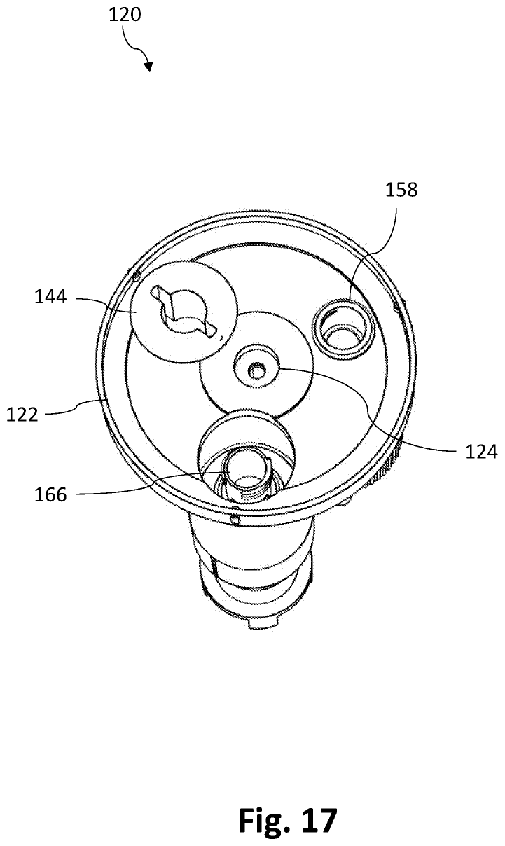

The front member can be rotated such that the catheter dock is selectively and consecutively accessible by the cap removal dock, fluid line dock, and the new cap dock. FIG. 12 shows the connector assembly with the front disc 112 hidden or removed to show a base 122 of the rear member 120. FIGS. 13 and 14 show the base 122 as having a planar member 121 and a skirt 123 extending upwards from the periphery of the planar member 121. The planar member 121 of the base 122, and the disc 112 can be of the same size. At the center of the planar member 121 can be seen a female mating member 124 that can couple with the male mating member 114 of the front member. The planar member of the base can have three apertures, wherein centers of the three apertures form vertices of an equilateral triangle. on the rear side of the planar member can be seen chambers or housings of the cap removal dock, fluid line dock, and the new cap dock. The three housings of the base can be integral with the planar member to form a single unit. The three housings can be continuous with the three apertures in the planar member as shown in FIG. 14. The housing 125 can be of the cap removal dock 140, the housing 126 can be for the fluid line dock 150, and the housing 127 can be of the new cap dock 160. FIG. 15 shows the connector assembly 100 without the base 122 and the three housings 125, 126, and 127, to show the position and arrangement of the cap removal dock 140, the fluid line dock 150, and the new cap dock 160. FIG. 16 shows the rear member 120 without the base 122 and the three housings, to show the position and arrangement of the cap removal dock 140, the fluid line dock 150, and the new cap dock 160. FIG. 17 shows a front side of the rear member and the three dock members i.e., cap removal dock 140, the fluid line dock 150, and the new cap dock 160 are also visible in FIG. 17.

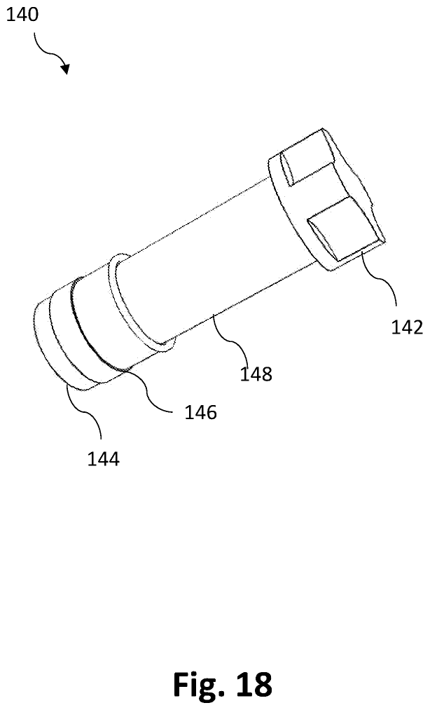

The housing 125 of the cap removal dock 140 can be coupled to the planar member of the base. The cap removal dock 140 can include a rod 148 that can be received within the housing 125. The distal end of rod 148 can have a head 142 while the proximal end of the rod can have a mouth 144. The mouth 144 of the rod 148 and the internal surface of the housing 125 can have suitable mating threads for securing the rod within the housing. O-rings, or a similar sealing mechanism, can be used to sealable couple the mouth 144 to the housing 125. FIG. 18 shows the O-ring 146, and a similar O-ring can be provided within housing 125. The O-rings or similar sealing mechanism can prevent the breach of contaminants during use. In use, when the catheter dock and the cap removal dock are aligned, and the catheter is secured into the catheter dock, the rod 148 can be pushed to clamp the cap of the catheter, the rod can then be turned to unfasten the cap of the catheter, and upon unfastening the cap of the catheter, the rod can be pulled to remove the cap of the catheter from the inlet port of the catheter. Alternatively, the catheter dock and the cap removal dock can be aligned default, and the inlet port of the catheter can be inserted into the catheter dock such that the cap of the catheter fits into the mouth of the rod. In case, rod 148 is secured in the housing 125 through mating threads, the mating threads can correspond to the mating threads of the cap of the catheter and the inlet port of the catheter. The catheter dock and the cap removal dock can be aligned default, and when the inlet port of the catheter is inserted into the catheter dock, the cap of the catheter fits into the mouth of the rod. After securing the catheter, the rod through the head of the rod can be manually turned anti-clockwise, which results in turning of the cap, and finally unfastening of the cap of the catheter from the inlet port, the rod 148 can also be simultaneously unfastened from the housing 125 when the cap of the catheter is unfastened from the inlet port. Thereafter, rod 148 can be pulled to withdraw the cap of the catheter from the inlet port. The mouth of the rod can snugly slide within the housing 125, such that to maintain sterility within the cap removal dock, similar to the plunger of a syringe.

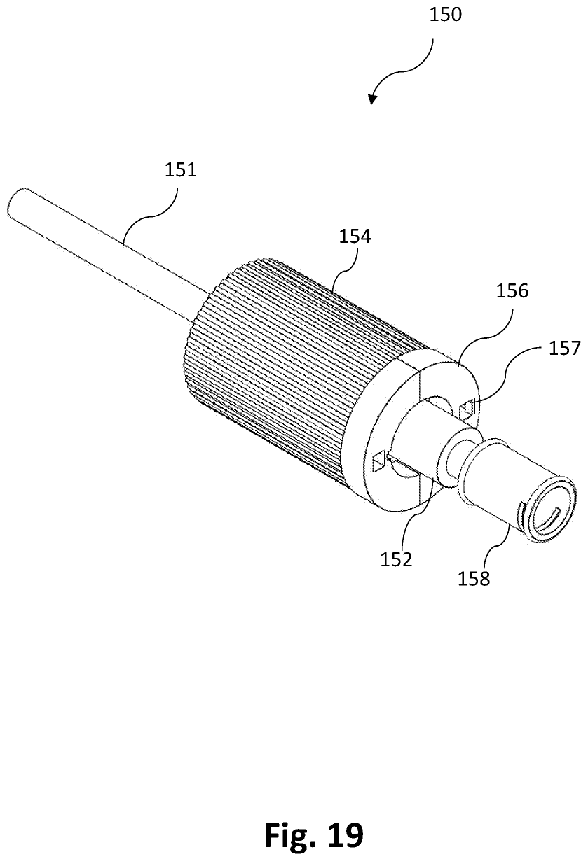

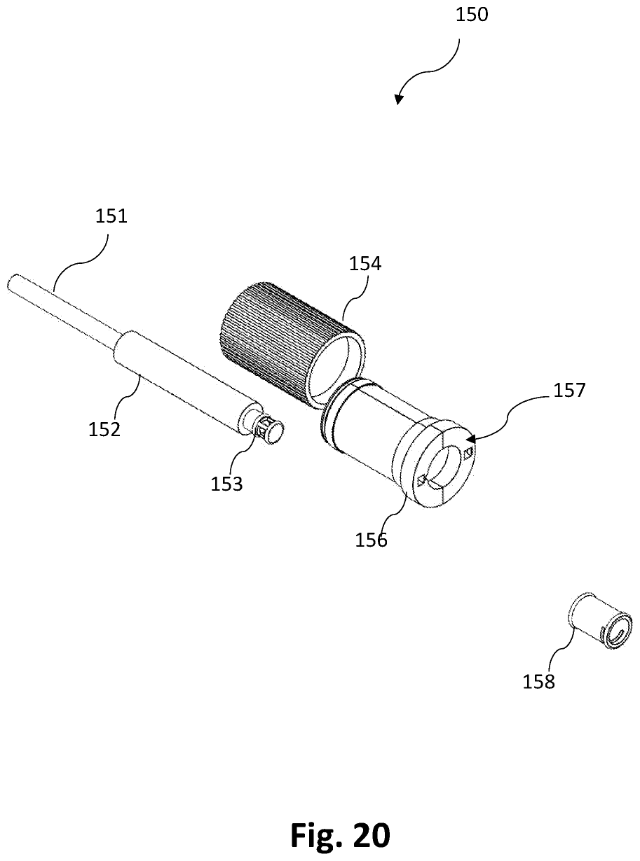

Referring to FIG. 19 which shows the fluid line dock 150 of the connector assembly 100. The housing 126 of the fluid line dock can be coupled to the planar member 121 of the base 122, as shown in FIG. 13. FIG. 20 shows an exploded view of the fluid line dock 150. The fluid line dock 150 can provide a sterile connection between fluid line 151 and the catheter. Once the cap of the catheter can be removed by the cap removal dock, the front member can be rotated to the next stop i.e., the fluid line dock 150. The fluid line dock 150 can include a cylindrical can-shaped outer piece 154. The outer piece 154 can have a washer base attached to stem 152 at the aperture's center. Outer-piece 154 has internal threads that mate with external threads of inner-piece 156 to allow the latter to be fastened onto the former. Inner-piece 156 is a two-piece component that forms a hollow cylindrical chamber when snap-fitted together. When inner-piece 156 is fastened onto outer-piece 154, the valve unit having a stem 152, stem-tip 153, and valve tip 158, runs parallel through inner-piece 156. The gap between inner-piece 156 interior and the exterior of stem 152 allow the stem 152 to run parallel through the opening of housing 126 and inner piece 156 to cover the exterior of housing 126. The fluid line dock 150 can include multiple O-rings (not pictured) placed around and inside both the outer-piece 154 and inner-piece 156, as well as housing 126, intended to prevent the breach of contaminants or prevent unwanted movement of assembly components when the connector assembly 100 is in use. On the front surface of inner-piece 156 is connector assembly lock (female) 157, which together with connector locks 128 in the base 122 (shown in FIG. 13), forms a snap-fitted lock preventing unwanted movement of the fluid line dock during use. Stem 152 is a solid tube connected to a fluid line 151 at its end. Stem 152 is a solid tube attached to the head of stem tip 153, the head of stem tip has side openings that allow fluid to pass through when unobstructed. Valve tip 158 includes one or more openings that enable a current of fluid to pass contingent upon the tip of stem tip 153 being fully inserted inside. When the assembly is activated for use, stem-tip 153 becomes fully inserted into valve tip 158, together forming a spool valve that initiates the flow of fluid between fluid line 151 and the inlet port of the catheter. Once, the fluid can be delivered, the fluid line dock can be unengaged, and the front member can be rotated to the next stop i.e., the new cap dock 160.

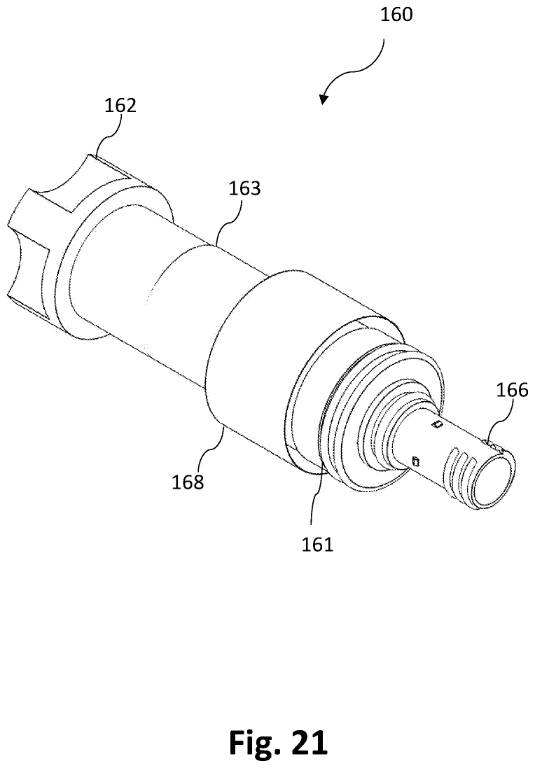

FIG. 21 shows the new cap dock 160 and the housing 127 of the new cap dock 160 can be coupled to the planar member 121 of the base 122 (shown in FIG. 13). The new cap dock 160 can include a rod 163, the rod 163 has a head 162, a new or replacement cap 166, an O-ring 161, a mouth 164, a position seal 168. The mouth 164 can clamp the new cap 166 wherein the new cap 166 is releasable from the clamp. The mouth 164 of the rod 163 can snugly slide within the housing 127, wherein O-ring 161 can function in conjugation with the O-rings inside housing 127 to prevent the breach of contaminants. The position seal 168 can be a plastic cover coupled to rod 163 and can secure rod 163 to housing 127.

In use, the user can remove the packaging of the connector assembly 100 and thereafter remove the seal of the catheter dock. The catheter dock can be aligned with the cap removal dock by default, and if not, the same can be aligned by rotating the front member. Thereafter, the user can fully insert the catheter into the catheter dock, wherein the cap of the catheter fits into the mouth of the cap removal dock. The catheter can be secured by the pin lock. The ring lock can be fastened to secure the pin lock. The rod of the cap removal dock can then be turned to unfasten the cap of the catheter. The rod of the cap removal dock can be turned counterclockwise several turns until the rod can freely be pulled back without resistance. Thereafter, the rod can be pulled, but the rod does not come out and the mouth of the rod can snugly remain fit within the housing to prevent the breach of contaminants. The rod of the cap removal dock in the pulled back state can be engaged with the mating threads in the housing of the cap removal dock wherein it remains for the rest of the treatment session.

Thereafter, the front member with the catheter and the cap of the catheter removed can be rotated to the next stop i.e., fluid line dock. The fluid line dock can be pushed forward along the housing of the fluid line dock, until lock 157 locks with lock 128. This action, which cannot be reversed, pushes the valve tip inside the inlet port of the catheter, and stem-tip inside the valve tip, enabling the free flow of fluid between fluid lines and the catheter. After treatment is complete, the user rotates the outer piece of the fluid line dock counterclockwise to unfasten it from inner-piece, subsequently releasing the valve stem, stem-tip, and valve tip from the inlet port of the catheter.

Thereafter, the front member can be rotated further to the next stop i.e., the new cap dock. The user can remove the position seal of the new cap dock. The rod of the new cap dock can then be pushed by the head into the housing of the new cap dock till the new cap is placed into the inlet port of the catheter. The user can then rotate the rod of the new cap dock clockwise to fasten the new cap into the inlet port. Once the new cap is secured to the catheter, the ring lock of the catheter dock can be unfastened, and the pin lock can then be released. The catheter can be pulled back from the catheter dock. All steps of the connection and disconnection can be performed under aseptic conditions in the enclosed environment of the connector assembly to ensure no contamination of the fluid pathway due to elements of human error including negligence in failure to follow treatment protocols or accidental touching of the fluid pathway.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A connector assembly for aseptically connecting and disconnecting a fluid line to a catheter, the connector assembly comprises:
a catheter dock, the catheter dock comprising:
a first housing configured to receive an inlet port of a catheter, the inlet port of the catheter has an opening, the opening secured by a catheter cap; and
a locking member configured to secure the inlet port within the catheter dock;
a cap removal dock comprising:
a second housing;
a first rod rotatably and slidably received into the second housing, the first rod has a mouth, the mouth of the first rod configured to releasably couple to the catheter cap while the catheter cap is fastened to the inlet port of the catheter; and
a fluid line dock comprising:
a third housing;
a valve unit encased within the third housing, the valve unit is in fluid communication with a fluid line, wherein the valve unit is configured to switch between an open state and a closed state, fluid from the fluid line flows freely from the valve unit in the open state, the fluid does not flow from the valve unit in the closed state,
wherein catheter dock is aligned with the cap removal dock such that the catheter cap can be removed by the first rod,
wherein the catheter dock is configured to aseptically switch from the cap removal dock to the fluid line dock,
wherein the fluid line dock is configured to be actuated to fluidly couple the valve unit and the inlet port of the catheter, while switching the valve unit from the closed state to the open state, resulting in a fluid communication between the fluid line and the catheter,
wherein the fluid line dock is configured to be separated from the inlet port of the catheter resulting in switching of the valve unit from the open state to the closed state.

2. The connector assembly according to claim 1, wherein the connector assembly further comprises:
a new cap dock, the new cap dock comprises:
a fourth housing;
a second rod slidably and rotatably received within the fourth housing, the second rod has a mouth; and
a new catheter cap releasably coupled to the mouth of the second rod,
wherein the catheter dock is configured to aseptically switch from the fluid line dock to the new cap dock such that the new cap dock and the catheter dock are aligned with each other,
wherein the new cap dock is configured to be actuated for fastening the new catheter cap to the inlet port of the catheter.

3. The connector assembly according to claim 1, wherein the connector assembly further comprises:
a front planar member, the front planar member has a front face and a rear face;
a rear planar member, the rear planar member has a front face and a rear face, the front planar member is rotatably mounted to the rear planar member, wherein the rear face of the front planar member faces the front face of the rear planar member,
wherein the first housing extends perpendicular from the front face of the front planar member,
wherein the second housing extends perpendicular from the rear face of the rear planar member,
wherein the third housing extends perpendicular from the rear face of the rear planar member, the second housing and the third housing are spaced apart from each other,
wherein the front planar member is configured to be rotated relative to the rear planar member for aseptically switching the catheter dock between the cap removal dock and the fluid line dock.

4. The connector assembly according to claim 2, wherein the connector assembly further comprises:
a front planar member, the front planar member has a front face and a rear face;
a rear planar member, the rear planar member has a front face and a rear face, the front planar member is rotatably mounted to the rear planar member, wherein the rear face of the front planar member faces the front face of the rear planar member,
wherein the first housing extends perpendicular from the front face of the front planar member,
wherein the second housing extends perpendicular from the rear face of the rear planar member,
wherein the third housing extends perpendicular from the rear face of the rear planar member,
wherein the fourth housing extends perpendicular from the rear face of the rear planar member, the second housing, the third housing, and the fourth housing are spaced apart from each other,
wherein the front planar member is configured to be rotated relative to the rear planar member for aseptically switching the catheter dock from the cap removal dock to the fluid line dock, and from the fluid line dock to the new cap dock.

5. The connector assembly according to claim 4, wherein the connector assembly further comprises a male fastening member and a female fastening member, wherein the male fastening member and the female fastening member are at centers of the front planar member and the rear planar member for rotatably coupling the front planar member to the rear planar member.

6. The connector assembly according to claim 5, wherein the male fastening member and the female fastening member are configured to halt rotation of the front planar member relative to the rear planar member at predefined intervals.

7. The connector assembly according to claim 6, wherein the male fastening member and the female fastening member are configured to limit the rotation of the front planar member relative to the rear planar member in one direction.

8. The connector assembly according to claim 7, wherein the predefined intervals are at 120 degrees.

9. The connector assembly according to claim 5, wherein the front planar member is sealably coupled to the rear planar member.

10. The connector assembly according to claim 4, wherein the first housing is integral with the front planar member, wherein the second housing, the third housing, and the fourth housing are integral with the rear planar member.

11. The connector assembly according to claim 1, wherein the connector assembly further comprises:
a pin lock comprising one or more pins, wherein the first housing has one or more apertures for the one or more pins to pass through into one or more apertures in the inlet port of the catheter.

12. The connector assembly according to claim 1, wherein the valve unit comprises:
a valve tip; and
a stem tip, the stem tip is in fluid communication with the fluid line, wherein the valve tip and the stem tip form a spool valve.

13. A method for aseptically connecting and disconnecting a fluid line to a catheter, the method comprising the steps of:
providing a connector assembly, the connector assembly comprises:
catheter dock comprising:
a first housing configured to receive an inlet port of a catheter, the inlet port of the catheter has an opening, the opening secured by a catheter cap, and
a locking member configured to secure the inlet port within the catheter dock,
a cap removal dock comprising:
a second housing,
a first rod rotatably and slidably received into the second housing, the first rod has a mouth, the mouth of the first rod configured to releasably couple to the catheter cap while the catheter cap is fastened to the inlet port of the catheter, and
a fluid line dock comprising:
a third housing,
a valve unit encased within the third housing, the valve unit is in fluid communication with a fluid line, wherein the valve unit is configured to switch between an open state and a closed state, fluid from the fluid line flows freely from the valve unit in the open state, the fluid does not flow from the valve unit in the closed state,
wherein the catheter dock is aligned with the cap removal dock such that the catheter cap can be removed by the first rod,
wherein the catheter dock is configured to aseptically switch from the cap removal dock to the fluid line dock,
wherein the fluid line dock is configured to be actuated to fluidly couple the valve unit and the inlet port of the catheter, while switching the valve unit from the closed state to the open state, resulting in a fluid communication between the fluid line and the catheter,
wherein the fluid line dock is configured to be separated from the inlet port of the catheter resulting in switching of the valve unit from the open state to the closed state;
inserting the inlet port of the catheter into the catheter dock;
upon inserting, securing the inlet port within the catheter dock by the locking member;
removing the catheter cap from the inlet port by turning the first rod to unfasten the catheter cap and upon unfastening the catheter cap, pulling the first rod to draw the catheter cap into the second housing;
upon removal of the catheter cap, switching the catheter dock to the fluid line dock; and
upon switching, when the catheter dock is aligned with the fluid line dock, actuating the fluid line dock to fluidly couple the valve unit and the inlet port causing the switching of the valve unit from the closed state to the open state and flowing of the fluid from the fluid line to the catheter.

14. The method according to claim 13, wherein the connector assembly further comprises:
a new cap dock, the new cap dock comprises:
a fourth housing,
a second rod slidably and rotatably received within the fourth housing, the second rod has a mouth, and
a new catheter cap releasably coupled to the mouth of the second rod,
wherein the catheter dock is configured to aseptically switch from the fluid line dock to the new cap dock such that the new cap dock and the catheter dock are aligned with each other,
wherein the new cap dock is configured to be actuated for fastening the new catheter cap to the inlet port of the catheter; wherein the method further comprises the steps of:
upon completion of administration of the fluid, separating the valve unit from the inlet port;
upon separating, switching the catheter dock to the new cap dock; and
fastening, the new catheter cap to the inlet port.
